(19) United States
(12) Patent Application Publication
(10) Pub. No.: US 2022/0236256 A1
(43) Pub. Date: Jul. 28, 2022

(54) MOBILE DEVICE BASED RAPID TEST SYSTEM, KIT, AND METHOD FOR PATHOGEN DETECTION

(71) Applicants: Erika Kay Cooper-Phillips, Roselle Park, NJ (US); Cory Bernard Phillips, Roselle Park, NJ (US); Valarie Thomas, Ann Arbor, MI (US); Latonia Harris, Yardley, PA (US)

(72) Inventors: Erika Kay Cooper-Phillips, Roselle Park, NJ (US); Cory Bernard Phillips, Roselle Park, NJ (US); Valarie Thomas, Ann Arbor, MI (US); Latonia Harris, Yardley, PA (US)

(21) Appl. No.: 17/649,266

(22) Filed: Jan. 28, 2022

Related U.S. Application Data
(60) Provisional application No. 63/199,834, filed on Jan. 28, 2021.

(51) Int. Cl. G01N 33/52; B01L 3/00; G01N 1/30; G01N 21/88; G06T 7/00

(52) U.S. Cl. CPC G01N 33/521; B01L 3/502715; G01N 1/30; G01N 21/8851; G01N 2021/6439; B01L 2300/0654; B01L 2300/168; B01L 2300/0809; G06T 7/0012

(57) ABSTRACT

A mobile device-based human pathogen rapid response diagnostic test system providing a partially disposable test kit and a system functioning within a mobile device software application. The diagnostic test system combines surfaces chemistry, thermochemical detection and automated histological digital imaging to reduce cradle-to-grave testing time relative to the state-of-the-art PCR methods.

### CROSS-REFERENCE TO RELATED APPLICATION

This application claims the benefit of priority of U.S. provisional application No. 63/199,834, U.S. provisional application number filed 28 Jan. 2021, the contents of which are herein incorporated by reference.

### BACKGROUND OF THE INVENTION

The present invention relates to medical testing, and more particularly to a mobile device-based rapid test system, kit, and method for pathogen detection.

There is an urgent need for medical tests for rapid detection of human pathogens, such as, for example without limitation, organisms belonging to the classifications of bacteria, fungi, protozoa, worm, virus, or prion. Viruses such as the human immunodeficiency virus (HIV), highly pathogenic influenza viruses (HPIV) and severe acute respiratory syndrome coronavirus II (SARS-CoV-2), also known as COVID-19 are several examples of pathogens for which personalized, rapid medical tests of adequate supply are needed. Currently, the latter virus is the root-cause of a worldwide pandemic.

By way of example, the pandemic of the coronavirus disease 2019, abbreviated as COVID-19 (“CO” stands for “corona”, “VI” for “virus”, and “D” for “disease”), revealed the global need for an invention of this nature. It should be understood that the following discussion regarding COVID-19 is not meant to limit the disclosure in any way to COVID-19, but rather COVID-19 is offered as a reference point to facilitate a broad understanding of the advantages of the present invention.

One major aspect of controlling the spread of COVID-19 or any pathogen among the public is having access to widely available, reliable, timely test methods. COVID-19 testing is currently being administered at an unacceptably slow pace often using invasive testing procedures. This slow pace of receiving reliable test results is unable to compete with the rate of transmission of the disease, and thus undermines testing efficacy in mitigating the spread. As of this writing, the U.S. has more deaths and positive cases than any other nation. Further, the limited testing capacity has negatively impacted the U.S. and global economy partly because of the delay in receiving the results; likewise, any subsequent strategically partial lockdown-restart attempts in the future will be hampered because of the lack of timely information on test results.

Common techniques for detecting SARS-CoV-2 and other pathogens employ reverse transcription polymerase chain reaction (RT-PCR) or “PCR” for short, a genetic material amplification technique. This technique requires that a patient generate enough of the test analyte (e.g., colloidal mucus, blood, saliva) of interest so that the genomic material can be reliably sequenced. Methods based on PCR are thus laborious and require significant time for sample processing using skilled technicians to extract and isolate the viral RNA first which is the most expensive and time-consuming step in the standard operating procedure. PCR based methods are additionally disadvantageous because of their dependence on expensive specialized chemical reagents that are in very limited supply while also being in high global demand. Hence, large, centralized laboratories are conducting most of the testing.

As the 2019 pandemic persisted, several companies and labs developed and marketed diagnostic tests that detected COVID-19 based on the SARS-CoV-2 virus' genetic material in a sample taken from the patient's nose or throat. The test analyte for these tests was most often collected on a nasal pharyngal swab inserted into the depths of the patient's nasal passage by a trained health professional. However, a key development over time was the capability to use samples harnessed by the patient (self-collection) themselves without any risk to the integrity or quality of the analyte. A few test methods using saliva as the test analyte were also developed in this manner.

All current PCR-based test methods employ information gained by multi-step sample processing that must be performed in a chemistry-enabled wet laboratory by highly trained personnel or by utilizing portable instrumentation functioning likewise so such processing can be achieved in a satellite location or on-site. The fastest response time for obtaining test measurement results only by any of the existing methods for pathogen identification thus far is on the order of 10-15 minutes. This does not include sample collection and handling nor any reagent and instrumentation preparation time.

Sampling is another concern and requires highly trained personnel to ensure specimen integrity. This task presently requires an uncomfortable, invasive upper respiratory nasal or blood sample from the patient. The use of nasopharyngeal swabs are the preferred materials for gathering upper respiratory samples, but these materials are produced by only a handful of manufacturers globally. Current testing processes serve mainly patients with apparent symptoms and are not administered frequently enough to find and monitor asymptomatic patients. Mass testing requires millions of tests per country per week in the most practical way according to epidemiologists.

Current PCR methods require extractions, gene identification, probes, and DNA amplification steps, which are complicated, time-consuming, and expensive. The result of current pathogenic detection processes, with their reliance on using specialized collection-instrumentation, the technical application of the collection-instrumentation, the proper isolation and handling of the collected sample prior to being tested (that may require physically shipping the collected sample) and communicating the test results accurately to possibly millions of individuals lack the requisite immediacy for tackling virulent, epidemical and pandemical pathogens.

Currently, all these widely used and popular test methods also invite logistical and administrative suboptimal outcomes when using mass testing as a tool to mitigate and control the spread of (potentially) epidemical and pandemical pathogens. When dealing with commercial, macroscale volumes of test specimens, testing errors, for instance, can be caused by poor sample handling, chain of custody mishaps, as well as, sample contamination causing false positives, or data mismanagement and quality.

The major limitation to controlling the spread of COVID-19 in the United States, as well as globally has been the absence of inexpensive, self-testing methods that non-invasively yet reliably and rapidly inform the end-user of the presence of the SARS-CoV-2 virus in their bodily fluid test analyte. Such a diagnostic testing solution has been provided in the following disclosure. The embodiments disclosed herein are easy to use, inexpensive and reduces test time, while permitting the end-user to remain in control of their medical information.

As can be seen, there is a need for a solution to these problems. A pathogen testing system and method is needed which is simple, effective, accurate, cost-effective and provides rapid results that enables efficient, collective mass testing. Moreover, the present invention, being a mobile-based, patient-driven testing system facilitates the efficacy and accuracy of mass testing; first, by not having the above-mentioned chain of custody problem. Additionally, the present invention obviates the logistical and bureaucracy-related problems of mass testing; for instance, anonymously registration and patient-identification issues can be mitigated as the same mobile device used to conduct the entirety of the testing can also be used to anonymously register and identify patients. Furthermore, the GPS and other mobile device locating functionality, say in a potential ‘outbreak hotspot’ or the like, can further aid an overseeing (governmental) body access risk and plan a coordinated, efficient response based on the statistical compilation that the present invention can enable when coupled to a global system of overseeing body.

### SUMMARY OF THE INVENTION

In one aspect of the present invention, a mobile device-based system for determining a presence of an analyte in a specimen, the system includes the following: a mobile device comprising a camera; a test kit comprising of a photo stage, wherein the photo stage operatively associates with the specimen; and both a system-software application (SSA) pair loaded on the mobile device, wherein the SSA is configured to receive a digital image captured by the camera, and wherein the SSA is configured to determine the presence of the analyte by applying an analytical algorithm to the digital image.

In one aspect of the system: a mobile device comprising a camera; a test apparatus comprising a photo stage, wherein the photo stage operatively associates with the specimen; and a systemic software application (SSA) loaded on the mobile device, wherein the SSA is configured to receive a digital image captured by the camera, and wherein the SSA is configured to determine the presence of the analyte by applying an analytical algorithm to the digital image, wherein the digital image captures the specimen operatively associated with the photo stage; including a chemically treated functionalized transparent test sheet, wherein said test sheet receives the specimen to operatively associate the specimen with the photo stage, wherein said test sheet has a specimen side and a reference side juxtaposed thereto, wherein the specimen side receives an entirety of the specimen; one or more infrared light emitting device (IR-LED) selectively transmitting infrared light against said test sheet, wherein the IR-LED is selectively arranged so that the test sheet absorbs infrared; an infrared sensitive lens optically associated with the camera, and wherein the specimen is saliva.

In another aspect of the present invention, the system further includes wherein the digital image captures the specimen operatively associated with the photo stage; and including a chemically treated functionalized transparent test sheet, wherein said test sheet receives the specimen in order to operatively associate the specimen with the photo stage, wherein said test sheet has a specimen side and a reference side juxtaposed thereto, wherein the specimen side receives an entirety of the specimen, wherein said test sheet has a specimen side and a reference side juxtaposed thereto, wherein the reference side remains clear of the specimen; further including one or more modulating infrared light emitting device (IR-LED) directing infrared light through a display of the photo stage on which said test sheet operatively associates; and an infrared sensitive lens optically associated with the camera, wherein the specimen is saliva.

In another aspect, the method for detecting a pathogen in a specimen by way of a mobile device includes the following: capturing, by way of a camera of the mobile device, a digital image of the specimen; and forming a decision as to a presence of the pathogen, by way of a software application loaded on the mobile device, in response to a multivariate statistical analysis of the digital image, wherein the multivariate statistical analysis comprises a correlation of a plurality of pixels of the digital image, wherein the correlation of the plurality of pixels comprises detection of a discrete spectrum captured in each pixel of the plurality of pixels; and selectively transmitting infrared light to the specimen upon or prior to capturing the digital image; treating the specimen with one or more thermochemical agents prior to capturing the digital image; carrying the specimen with a transparency, wherein the one or more thermochemical agents occupy the transparency, wherein the transparency comprises a specimen side and a reference side, and wherein only the specimen side supports the specimen; and histologically staining the specimen prior to capturing the digital image; treating the specimen with one or more surface agents prior to capturing the digital image, wherein the one or more surface agents promote the multivariate statistical analysis, wherein the camera has an infrared sensitive lens.

These and other features, aspects and advantages of the present invention will become better understood with reference to the following drawings, description and claims.

### BRIEF DESCRIPTION OF THE DRAWINGS

FIG. 1 is a flow chart of an exemplary embodiment of the present invention, showing the general diagnostic test kit procedure and system.

FIG. 2 is a schematic view of an exemplary embodiment of the present invention showing an end-user interacting with a mobile device via a touch display user interface.

FIG. 3 is a schematic view of an exemplary embodiment of the present invention showing a mobile device displaying a system-software application (SSA) enabled user interface representing a guided instructions/checklist.

FIG. 4 is an exploded perspective view of an exemplary embodiment of the present invention showing a partially disposable test kit showing a layout of the fully disposable transparent functionalized test sheets and permanent photo stage.

FIG. 5 is a schematic view of an exemplary embodiment of the present invention illustrating usage of the partially disposable test kit, comprised of a layout of the fully disposable transparent functionalized test sheets and permanent photo stage.

FIG. 6 is a schematic view of an exemplary embodiment of the present invention illustrating end-user interacting with the mobile device and the partially disposable test kit to capture digital images for system-software application (SSA) uploading and automated processing.

FIG. 7 is a flow chart of an exemplary embodiment of the present invention showing the key automated digital imaging processing steps required to confirm the presence of a pathogen on the associated captured image 13 of the specimen side/reference side 10 of the test sheet.

FIG. 8 is a perspective view of an exemplary embodiment of the present invention of the source of IR emission within the photo stage 7 as part of the test apparatus/partially disposal test kit 15, with a portion thereof enlarged to show detail.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention may provide a mobile device-based test system, kit, and method for the rapid detection of the presence of a pathogen. The disclosure embodies a method to facilitate rapid mass testing for mitigating epidemical and pandemical pathogenic outbreaks. The present invention relates to the use of a mobile device (may include common devices, for example without limitation, such as smartphones, smartwatches, tablets, netbooks, personal digital assistants, MP3 players, cell phones, e-book readers, and the like) to facilitate a non-invasive self-testing kit to identify the presence of a pathogen, thereby enabling a user to test the user's own bodily fluid such as saliva, blood, breath, urine or sweat as the test analyte. In some embodiments, the present invention is a public health assessment tool. Pathogens of interest are those that cause infectious disease and could be one or more organisms belonging to the classifications of bacteria, fungi, protozoa, worm, virus, or prion. The human immunodeficiency virus (HIV), highly pathogenic influenza viruses (HPIV) and severe acute respiratory syndrome coronavirus II (SARS-CoV-2), also known as COVID-19, are several examples of pathogens that the present invention would detect from an aliquot of body fluid.

In one aspect, the test system, kit, process and method of the present invention may combine surface chemistry, thermochemical detection and automated histological digital imaging to improve testing accuracy and reduce time. Advantageously, the present invention may provide delivery of results in minutes and may require no “designated safe-testing site”, commercial lab with expensive lab equipment, reagents, and/or specialized personnel. The present invention may provide a non-invasive testing option, requiring only a saliva sample from an end-user which may be provided via a disposable test strip/sheet (for collection of a sample such as, for example without limitation, a saliva sample or sample of other bodily fluid). The present invention offers convenient use and may only require a mobile device, a test sample collection disposable test strip/sheet kit on the order of a small wallet, business or credit card, and a photo stage.

In another aspect the present invention offers reliability and accuracy. The present invention may be used to test saliva, which has recently been shown to be more consistent with respect to data quality in PCR and commensurate in terms of free viral concentration relative to nasal swab samples. Further, the present invention may provide testing that is accurate, rapid, inexpensive, and easy-to-use, with no large-scale, complex, significant grassroots manufacturing effort required for scale-up to the public. The integrity of the sample may be preserved within the invention procedures. End-users of the present invention may take the test as frequently as necessary, like a home pregnancy test, and not worry about their DNA being handled by unknown third parties. End-users of the present invention are in total control of their medical information.

The system may include at least one computer (mobile device) with a user interface available to an end-user. The computer may include at least one processing unit coupled to a form of memory that may be accessed. The computer may include, but may not be limited to, a microprocessor, a server, a desktop, a laptop, a mobile device and a smart device, such as, a tablet, smart phone or smart watch. The computer may include a program product including a machine-readable program code for causing, when executed, the computer to perform steps. The end-user may engage this program product via the user interface. The program product may include software which may either be loaded onto the computer or accessed by the computer. The loaded software may include an application on a mobile smart device. The software has the ability to interact with other software applications on the smart device such as, a camera and a photo library. The software may be accessed by the computer using a web browser capable of transferring/receiving data and information seamlessly. The computer may access the software via the web browser using the internet, extranet, intranet, host server, internet cloud and the like via the wireless communication protocols available on the mobile device. The calculations, data processing and data refinement tasks required for reporting results back to the end-user through the user interface may be automated.

The ordered combination of various ad hoc and automated tasks in the presently disclosed system-software application (SSA) may necessarily achieve technological improvements through the specific processes described more in detail below. In addition, the unconventional and unique aspects of these specific automation processes represent a sharp contrast to merely providing a well-known or routine environment for performing a manual or mental task.

This invention may assist federal, state and local municipalities along with public/private for-profit and non-profit corporations and institutions in their public health efforts with detecting, tracking and ultimately containing innumerable viral contagions by providing a non-invasive, widely accessible, home-based, point-of-care, rapid test method equipped with available capacity at all times. Users of the present invention no longer need to wait hours and days for results, and the test can be administered as frequent as necessary enabling families and friends to congregate, workplace activity to resume, key events, meetings, conferences, air travel and mass transit to ensue with a low risk of transmission. The present invention allows for “minute by minute” testing and monitoring thus, decelerating the spread while accelerating economic re-start.

The present invention differs from and distinguishes over what currently exists. For instance, as of this writing, the most widely used “gold-standard” of COVID-19 test methods are time consuming and based on bulk fluid-phase, homogenous, reverse transcriptase-polymerase chain reaction (RT-PCR or “PCR”) chemistry where the extracted genes of the COVID-19 virus RNA present in the patient are converted to DNA, tagged with special probes (e.g., fluorescent agents) and then amplified by building more DNA to increase detection sensitivity. The invention presented here is a heterogenous surface chemical binding method coupled with a digital scheme requiring no extractions, no gene identification, no probes and no DNA amplification steps.

Other test methods and processes based on PCR test methods need highly trained, skilled technicians to extract and isolate the viral RNA first which is the most expensive and time-consuming step in the standard operating procedure. The overall process takes too long due to the heating and cooling cycles required to amplify the DNA. Further, the equipment is not widely available because it is quite expensive and requires special, proprietary reagents that are often limited in supply and non-locally sourced. Hence, large, centralized laboratories are conducting most of the testing.

The present invention provides an unprecedented solution to this problem by combining IR interaction, surface chemistry, and automated digital imaging to reduce cradle-to-grave testing time relative to the state-of-the-art PCR methods. The surface chemistry may include some histological functionality to enhance the automated digital imaging task. This invention delivers results on the order of minutes, requiring no designated safe testing site, commercial lab with expensive lab equipment, reagents or personnel and is non-invasive, requiring only a saliva sample from the end-user, a mobile device and a partially disposable test kit. Further, this invention is accurate, rapid, inexpensive, and easy-to-use, with minimum or no large scale, complex, significant grassroots manufacturing effort required for scale-up to the public. The integrity of the sample is accomplished and maintained within the invention procedures. End-users may take the test as frequently as necessary, like a home pregnancy test, and not worry about their DNA being handled by unknown third parties. End-users are in total control of their medical information.

The present invention can detect other pathogens and SARS type viruses. The present invention may have further applicability to detection of other SARS-based or related viruses, antibody testing, and any other rapid testing application.

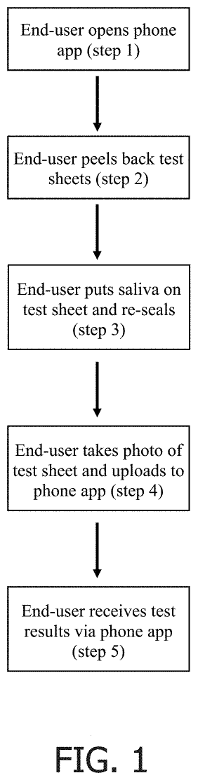

Referring now to FIGS. 1 through 8, incorporated herein by reference in their entirety, the present invention may include a rapid, mobile device-based pathogen test and detection system and method. In an exemplary embodiment, the present invention may include one or more of the following elements or components, method or process steps, and software components, and combinations thereof.

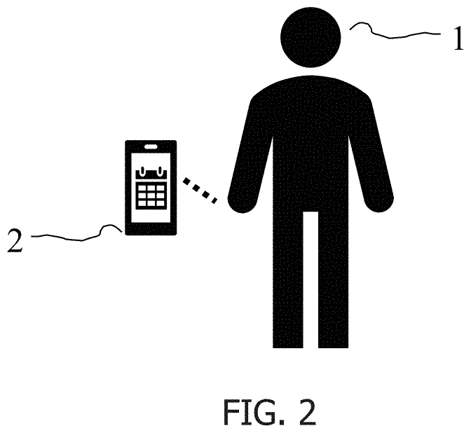

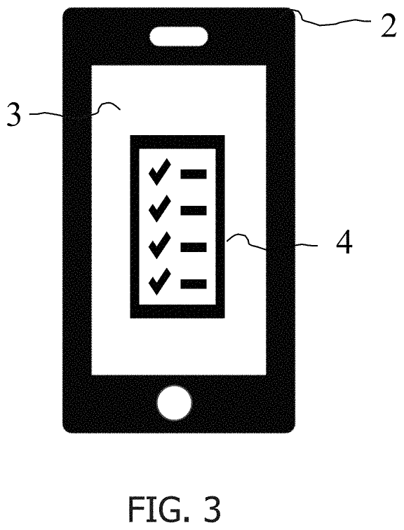

Step 1 of the process includes an end-user 1 engaging/opening the SSA pathogen test application 3 on a mobile device 2. The pathogen test application 3 may display instructions for the end-user 1 in the form of a guideline/checklist 4 as shown in FIGS. 2 and 3.

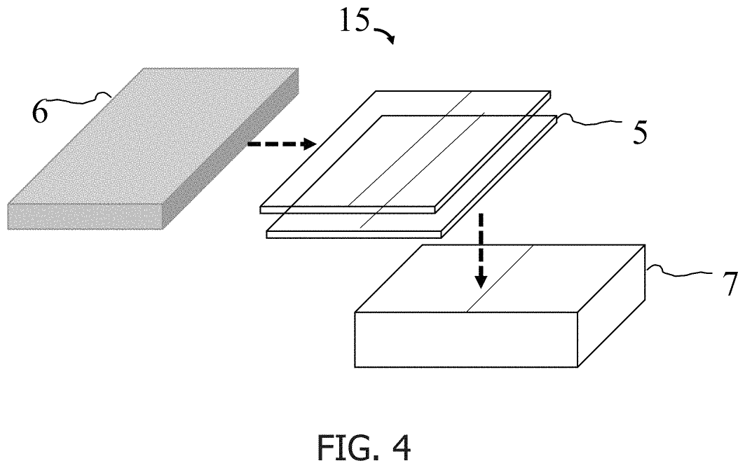

In step 2, the end-user 1 may be directed to open a fresh, unsealed, unused chemical treated functionalized transparent test sheet 5 from packaging 6 and place on the photo stage 7 as shown in FIG. 4. The test sheet 5 may have a specimen side 9 and a juxtaposed reference side 10. The chemical treated functionalized transparent test sheet 5 may be translucent. The sheet material may be a film, and so the test sheet may be known as a transparency.

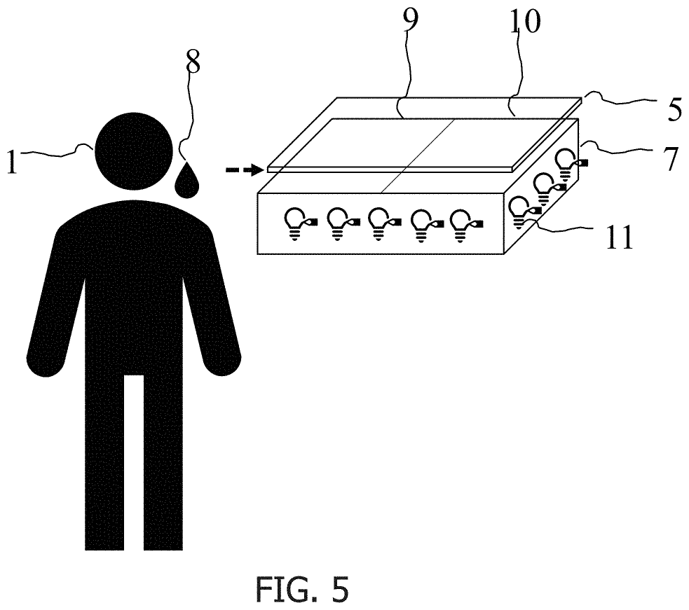

During step 3, the end-user 1 deposits a specimen sample (including but not limited to saliva 8) on the specimen side 9, keeping the reference side 10 free of specimen deposits. In some embodiments, the end user 1 may bring the entire test apparatus 50 close to the end user's own mouth and applies saliva 8 on the specimen side 9 and re-seals, keeping the reference side 10 clean and dry as shown in FIG. 5.

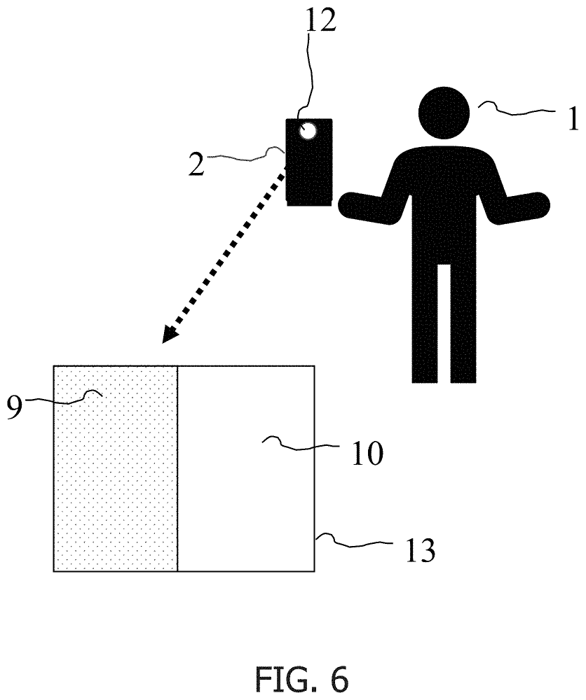

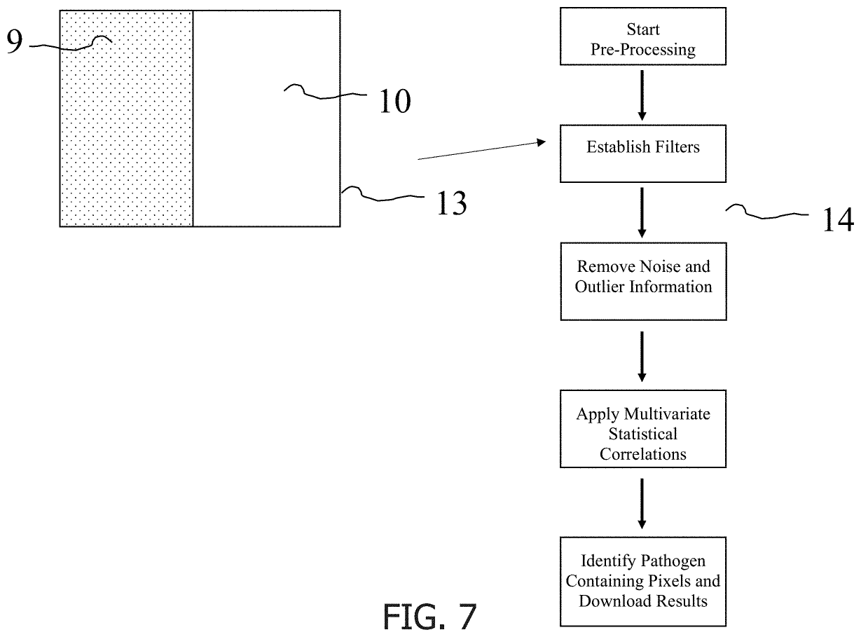

Step 4 may include the end-user 1 turning on the array of near IR-LED lights 11 within the photo stage 7 then taking a digital image 13 of the entire test sheet 5—both specimen 9 and reference 10 sides—using an image capturing device (camera) on the mobile device 2 wherein the camera has a standard factory-installed infra-red (IR) sensitive lens 12 as shown in FIG. 6. The array of near IR-LEDs is arranged in the exact configuration between the specimen 9 and reference 10 sides, and can vary in spatial density, as well as peak emission wavelength. The captured digital image 13 is then uploaded into the pathogen test application 3 where the visual spatial and spectral information are deconvoluted an organized in a manner like a BIL, BIP or BSQ file format for a multivariate image with wavelength channels. The pathogen test application 3 then proceeds to execute an automated hyperspectral-like imaging processing analysis 14 of the captured digital image 13 identifying the presence of any pathogens or viral bodies through visual and multivariate statistical algorithms as shown in FIG. 7. The difference between the multivariate images created from specimen 9 and reference 10 sides is used correlate to pathogen presence during the spectral analysis shown in FIG. 7.

The photo stage 7 may include a glass or transparent platen on which the test sheet 5 is placed, and that a light (as well as infrared and other electromagnet waves) source is below the platen. However, it is to be understood that the term “photo stage may include any device that supports the test sheet as it projects one or more electromagnetic wave types against the test sheet 5 when the user is capture the digital image 13. To that end, the photo stage 7 may provide the electromagnetic wave source in other locations relative to the platen (or equivalent), as opposed to being limited to underneath. And the electromagnetic wave could be bounced, reflected, or otherwise have a modified path length prior to it contacting the test sheet, and thus the electromagnetic wave source being, in part, absorb in the test sheet 5. The photo stage 7 may be simply any means of getting electromagnetic waves (e.g., infrared) absorbed into the test sheet 5 during or before the capture of the digital image 13.

In step 5, once the multivariate, hyperspectral-like imaging processing analysis 14 is completed the test results are downloaded back through the pathogen test application 3 to the end-user 1 on their mobile device 2.

In certain embodiments, it is critical that steps 1-5 are completed sequentially to accomplish the acquisition and analysis of saliva-based test specimen images which is the core of this invention.

In an exemplary embodiment, the present invention may work in the following manner. The present invention provides a testing process and method based on advanced visual identification techniques for the presence of pathogenic particles in saliva 8 via on-line automated processing of captured digital image 13 taken from a (possibly common) digital camera with an IR-sensitive lenses 12.

Pathogenic identification via multivariate image analysis may be enhanced by coupling several surface chemistry techniques:

1) detection of infra-red (IR) signal changes due to exothermic thermochemical binding events involving specific gene sequences on viral RNA such as, the S1 and S2 regions of COVID-19 characteristic spiked glycoproteins; S1+2*→S1*+S2* where * denotes the active sites on the surface of the chemical treated functionalized transparent test sheet 5 binding to various regions of the pathogen/analyte and releasing heat;

2) visual detection of viral color bodies facilitated by targeted histologic chemical staining manifesting in the spatial data of the multivariate image; and/or

3) a combination of the former and latter aforementioned techniques. For instance, the histological chemical staining enhances the efficacy of the spectral analysis of the thermochemical binding events (by, in some embodiments, excluding things except the pathogenic material when being digitally processed). Likewise, there are forms of applied surface chemistry that facilitates the mathematical modeling underlying the multivariate digital imaging processing.

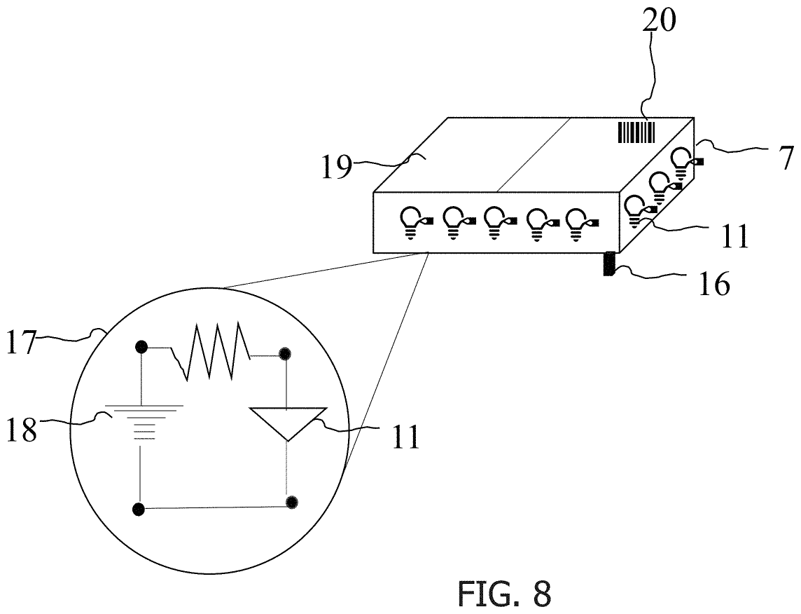

In this diagnostic testing method and process, end-users 1 may administer the procedure at home or “on the go” without the need for a healthcare professional. All the materials required for the diagnostic test method may be contained in a partially disposable test kit 15. The partially disposable test kit 15 may include: a package of individually wrapped, chemically treated functionalized transparent test sheets 5 resembling thin (<10 mil) rectangular plastic shims or films; and a photo stage 7 to assist with capturing digital photo images. As shown in FIG. 8, The photo stage 7 may include an on/off switch 16 to provide battery power 18 to the miniature, near IR-LED light array 11 and corresponding circuits 17 housed underneath translucent photo staging display 19. Each photo stage 7 may be tagged with a bar code 20 stamped on the photo staging display 19 capable of keeping results organized for monitoring by the end-user 1.

In some embodiments, the testing apparatus may be battery powered. In other embodiments the testing apparatus may be photovoltaic (solar powered).

### Use of Partially Disposable Test Kit

First, the end-user 1 places a test sheet 5 on the photo stage 7 which can have a surface area range and shape as small as business card (1″ W×2″ L) to as large as a piece of chromatography paper (½″ W×5″ L). The end-user 1 then administers a small volume (<5 cc) of saliva 8 onto the specimen transparent test sheets 5 that is coated/treated with a variety of solid, surface-bound reagents with binding affinity toward specific pathogens, such as COVID-19. If desired, these surface reagents could be equivalent or comparable to those used in the PCR technique such as, enzymes (for example without limitation, reverse transcriptase) or cheaper more widely available reagents supporting different or novel chemistry (for example without limitation, lectin).

Once the saliva 8 contacts the coated/treated transparent test sheet or sheets 5, then several interactions and chemical transformations may take place if distinguishing pathogenic markers such as, viral RNA are present. Depending on the application, the surface reagents could stay bound to test sheet 5 or become dissolution active when brought in contact with saliva 8. In some embodiments, the surface-bound reagents may have a binding affinity toward a selected viral RNA.

One key interaction may involve exothermic binding (>−80 kJ/mol) of viral specific reagents such as, proteins, enzymes, monoclonal antibodies, probes, and lectin, with the spiked glycoproteins present on the surface of viral macromolecules like COVID-19 and RNA genome. It is predicted that the viral RNA load in saliva 8 may be on the order of 1×106 copies/cc with the average molecular diameter ranging from 60-140 nm and up to 30,000 nucleotides. Each spiked glycoprotein is considered a trimer in the pre-fusion state. As an example, there are 100 spikes per COVID-19 viral particle. The presence of these thermochemical binding events may vary depending on the surface chemistry and concentration. More importantly, the occurrence of these exothermic binding events may be detected as changes in the modulating shifts in absorption intensity of a saliva specimen 8 in the presence of an IR-LED light source array 11 relative to a similar reference source 10 free of viral bodies. For instance, the reaction between the pathogenic material and the IR may result in heat release (or a creation of a heat sink) that can be detecting during the subsequent spectral analysis (multivariate digital imaging processing) step.

A second key chemical interaction may involve specialized color inducing nucleic acid stains, enzymes, and dyes such as, hematoxylin and eosin (H&E) that bind to both the acidic and basic regions of the viral particles causing histologic staining and enabling visual observations to be made through digital imaging methods. Further, some of these interactions may contribute positively to the thermochemical interactions discussed above improving detection efficacy. The IR-LED light source array 11 will assist in the visualization with tiny specs of stained viral bodies possibly even visible to the naked eye at this stage with magnifications >20×.

Both types of chemical interactions may take place within minutes of contacting pathogenic disease, such as but not limited to COVID-19, containing saliva specimens making this a rapid technique.

After several minutes pass allowing sufficient time for both the thermochemical and histologic interactions to play out within the saliva specimen 8, the end-user is then ready to take a digital image 13 preferably using the IR-enabled camera lens 12 commonly available on most cell phones 2. (This is not the IR attachment technology that must be purchased to take thermographic images, however, that technology is compatible with this method—therefore, in some embodiments, the kit, system, and method may optionally include this as a component.) This type of cellular phone camera lens 12 is already part of the standard manufactured features for many cell phones 2 on the market. There may or may not be visual evidence of viral particles to the naked eye at this point of the test which is why this multivariate digital imaging processing step is essential. Once the captured digital image 13 is taken then it may be uploaded to the system executing the automated multivariate digital processing application that filters out all of the imaging noise and interferences using automated hyperspectral-like imaging methods 14. Once the original digital photo image 13 is pre-processed, modelled statistically and re-imaged then the clear, irrefutable presence of viral bodies becomes apparent and may be quantified and characterized based on physical, as well as geometrical attributes. Because the array of IR LED lights 11 may be configured in different patterns and different wavelengths (700-900 nm), a discrete change in IR signal relative to the reference signal is produced and captured in each pixel. This discrete change once simulated into specific wavelength channels, as well as the histologic staining spatial information comprises this multivariate statistical imaging method that is correlating pixels. This automated digital processing step 14 takes place in minutes and may be facilitated by use of the mobile device-based software applications 3. Once the determination of the virus particles has been made, the results of the digital imaging analysis will be communicated rapidly back to the end user as “negative” or “positive” within the pathogen test application 3 for the presence of selected pathogenic particles. End-users 1 may be, through a protocol to control the spread of the selected pathogen, encouraged to perform the analysis at least three times with each testing round to gain statistical confidence if desired.

In an exemplary embodiment of the present invention, the system may be made in the following manner. Mass assembly for the hardware components of the partially disposable testing kit of this invention namely, treated functionalized transparent test sheet 5, photo stage 7, photo staging display 19, IR-LED light cells/arrays 11, may be accomplished by making small modifications to existing manufacturing processes. For instance, the current manufacturing processes for clear adhesive tape, clear plastic films and coatings, chromatography paper or home pregnancy strip paper may be modified to include the chemical functionalization necessary to carry out the surface interactions on the test sheets 5 required in this invention.

The test sheets 5 may be made by mixing an adsorbent mixture (e.g., enzyme, lectin, antibodies) and a small amount of inert binder and water. This mixture is spread and coated as a slurry or a clear mixture of varying thickness on an un-reactive carrier sheet, film or chromatography type-paper then dried. Another clear mildly adhesive clear film is placed over the surface of dry treated sheet for protection and quality control integrity. The final test sheets 5 may be packaged individually and grouped into specific denominations (e.g., a sheet pack of one dozen).

The photo stage 7 may be a rigid, box-type plastic housing for the near IR-LED light cell array 11 and control circuitry 17. These types of rigid packaging containers are in abundant supply across the extruded plastics market. The photo staging display 19 may be an impact polymer composite of a particular tint or color that is translucent allowing for light to go through. The near IR-LED light cell array/circuitry 11/17 and circuits are already in mass production used abundantly in in-home remote controller applications.

From the software perspective, an automated multivariate imaging algorithm 14 must be written allowing for uploaded images 13 to be recognized, picked up in a dedicated server as “jobs”, pre-processed, processed, and validated against existing correlations or statistical models. This algorithm 14 must be written to work within the cellular phone application environment.

All three of the aforementioned elements are necessary for the invention to work and give end-users 1 the positive or negative results.

The component or elements of this invention can be reconfigured or interchanged to perform an identical or similar function. It is specifically configured for COVID-19. However, the configuration can be adjusted to identify other SARS related strains as well as any other identifiable pathogen.

The system and method of the present invention may have additional applications. The components or elements of the system, steps of the method or modules of software elements may be configured, reconfigured or interchanged to perform an identical or similar function. The system, method and software are configured to apply to COVID-19 but may be applicable to any pathogen. The configuration may be adjusted to identify other SARS related strains or other related pathogens. The system, method and software may be applicable to provide any rapid, home-based, pathogen or viral detection or antibody testing systems or methods.

In an exemplary embodiment, the present invention may be used in the following manner. The person that uses the present invention would be able to solve several testing problems that are currently pervasive during the COVID-19 pandemic, and thus threaten future attempts to mitigate other pathogenic epidemics. This invention solves the issue of long waits for test results. The test results will be available for the end-user in a matter of minutes. The present invention also alleviates the issue of single contingency supply risk. The present invention does not rely on specific proprietary brand of PCR reagents that are in short supply. The present invention also alleviates the dependence of the specific nasal swab used for the current testing used by large, centralized labs. The present invention also solves the problem of DNA specimens being handled by parties other than the end-user. The present invention is also non-invasive. The costs associated with the disclosure make it inexpensive compared to the current PCR tests. The present invention allows organizations (e.g., corporate, government, university, non-profit etc.) communities and families to know immediately if their members are COVID-19 positive thereby alleviating additional exposure of the virus to other members and providing for more control of the spread of the virus and disease, not only through test confirmation, but also by the testing method itself. Additionally, the disclosure could be utilized for other home-based viral detection methods or for antibody testing. Also, the present invention can detect the presence of other SARS based viruses or related pathogens.

In summary, in an exemplary embodiment, the present invention may provide a mobile device (such as, for example without limitation, a cellular smartphone) based human pathogen (such as, for example without limitation, COVID-19) rapid response diagnostic test system comprising a partially disposable test kit and a system functioning within a mobile device software application.

The present invention may provide a mobile device-based test system, kit, process and method for the rapid, detection of the presence of one or more human pathogens.

The test system and method of the present invention may combine IR interaction, surface chemistry, and automated digital imaging to improve testing accuracy and reduce time. The test of the present invention may deliver results in minutes, requiring no designated safe testing site, commercial lab with expensive lab equipment, reagents or personnel. The test of the present invention may be non-invasive, requiring only a saliva sample from the end-user, a mobile device, such as a cellular smartphone, and partially disposable test kit.

Further, the present invention is accurate, rapid, inexpensive, easy-to-use with minimum manufacturing required for scale-up to the public. The integrity of the sample is accomplished and maintained within the invention procedures. End-users may take the test as frequently as necessary, similar to a home pregnancy test, and not worry about their DNA being handled by unknown third parties. The end-user is in total control of their medical information.

The computer-based data processing system and method described above working in conjunction with the mobile device software application is for purposes of example only and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A mobile device-based system for determining a presence of an analyte in a specimen, the system comprising:
a mobile device comprising a camera;
a test apparatus comprising a photo stage, wherein the photo stage operatively associates with the specimen; and
a systemic software application (SSA) loaded on the mobile device, wherein the SSA is configured to receive a digital image captured by the camera, and wherein the SSA is configured to determine the presence of the analyte by applying an analytical algorithm to the digital image.

2. The system of claim 1, wherein the digital image captures the specimen operatively associated with the photo stage.

3. The system of claim 2, further comprising a chemically treated functionalized transparent test sheet, wherein said test sheet receives the specimen to operatively associate the specimen with the photo stage.

4. The system of claim 3, wherein said test sheet has a specimen side and a reference side juxtaposed thereto, wherein the specimen side receives an entirety of the specimen.

5. The system of claim 4, further comprising one or more infrared light emitting device (IR-LED) selectively transmitting infrared light against said test sheet.

6. The system of claim 5, wherein the IR-LED is selectively arranged so that the test sheet absorbs infrared.

7. The system of claim 6, further comprising an infrared sensitive lens optically associated with the camera.

8. The system of claim 7, wherein the specimen is saliva.

9. The system of claim 8, wherein the analytical algorithm comprises hyperspectral imaging processing analysis, by way of the SSA, against to the digital image.

10. A method for detecting a pathogen in a specimen by way of a mobile device, the method comprising:
capturing, by way of a camera of the mobile device, a digital image of the specimen; and
forming a decision as to a presence of the pathogen, by way of a software application loaded on the mobile device, in response to a multivariate statistical analysis of the digital image.

11. The method of claim 10, wherein the multivariate statistical analysis comprises a correlation of a plurality of pixels of the digital image.

12. The method of claim 11, wherein the correlation of the plurality of pixels comprises detection of a discrete spectrum captured in each pixel of the plurality of pixels.

13. The method of claim 12, further comprising selectively transmitting infrared light to the specimen upon or prior to capturing the digital image.

14. The method of claim 13, further comprising treating the specimen with one or more thermochemical agents prior to capturing the digital image.

15. The method of claim 14, further comprising carrying the specimen with a transparency, wherein the one or more thermochemical agents occupy the transparency.

16. The method of claim 15, wherein the transparency comprises a specimen side and a reference side, and wherein only the specimen side supports the specimen.

17. The method of claim 16, further comprising histologically staining the specimen prior to capturing the digital image.

18. The method of claim 17, further comprising treating the specimen with one or more surface agents prior to capturing the digital image, wherein the one or more surface agents promote the multivariate statistical analysis.

19. The method of claim 18, wherein the camera has an infrared sensitive lens.
